(19) United States
(12) Patent Application Publication
(10) Pub. No.: US 2026/0364563 A1
(43) Pub. Date: Nov. 19, 2026

(54) MXene Sorbent For Removal Of Small Molecules From Dialysate

(71) Applicants: Drexel University, Philadelphia, PA (US); University of Brighton, Brighton (GB)

(72) Inventors: Yury Gogotsi, Warminster, PA (US); Sergey Mikhalovsky, Brighton (GB); Susan R. Sandeman, Brighton (GB); Babak Anasori, Fisher, IN (US); Fayan Meng, Philadelphia, PA (US)

(21) Appl. No.: 19/649,392

(22) Filed: Apr. 16, 2026

Related U.S. Application Data
(63) Continuation of application No. 18/353,257, filed on Jul. 17, 2023, now Pat. No. 12,629,652.
Application No. 18/353,257 is a continuation of application No. 17/670,716, filed on Feb. 14, 2022, now Pat. No. 11,772,066.
Application No. 17/670,716 is a continuation of application No. 16/633,773, filed on Jul. 12, 2018, now Pat. No. 11,278,862.
(60) Provisional application No. 62/539,715, filed on Aug. 1, 2017.

(51) Int. Cl. B01J 20/02; A61M 1/16; B01D 15/08; B01J 20/28; C02F 1/28; C02F 101/38

(52) U.S. Cl. CPC B01J 20/0211; A61M 1/1656; B01D 15/08; B01J 20/0218; B01J 20/0274; B01J 20/28052; C02F 1/281; B01J 2220/42; C02F 2101/38; C02F 2201/006

(57) ABSTRACT

The present disclosure is directed to methods for scrubbing low levels of urea from aqueous solutions such as a dialysate from dialysis, and including blood and blood products, and devices capable of employing these methods.

### CROSS REFERENCE TO RELATED APPLICATIONS

This application is a continuation of now-allowed U.S. patent application Ser. No. 18/353,257, filed Jul. 17, 2023; which is a continuation of U.S. patent application Ser. No. 17/670,716, filed Feb. 22, 2022 (now U.S. Pat. No. 11,772,066, issued Oct. 3, 2023); which is a continuation of U.S. patent application Ser. No. 16/633,773, filed Jan. 24, 2020 (now U.S. Pat. No. 11,278,862, issued Mar. 22, 2022); which is the National Stage Application of International Patent Application No. PCT/US2018/041786, filed Jul. 12, 2018; which claims priority to U.S. Provisional Application No. 62/539,715, filed Aug. 1, 2017. All foregoing applications are incorporated herein by reference in their entireties for any and all purposes.

### TECHNICAL FIELD

The present disclosure is directed to the purification of blood and blood products, for example for the treatment of renal failure or chronic kidney failure.

### BACKGROUND

Chronic renal failure or chronic kidney failure is a condition when a large number of compounds that are normally excreted by the kidneys remain in the body. Urea is one of the crucial nitrogen-containing metabolites in biological system. However, when the kidneys fail, it remains in the body. Increased blood urea nitrogen (BUN) is associated with kidney disease or failure. Once kidney function drops to life threatening levels, hemodialysis is used as a proven, safe procedure to remove excess fluid, electrolytes and the majority of small, water soluble molecules including urea by diffusion through a semipermeable porous membrane into the dialysate fluid. However, hemodialysis is by no means optimized. It is time-consuming, cumbersome and mobility-restricting. Miniaturization of the presently employed hemodialysis systems into high effective, portable and low-cost devices (i.e., a Wearable Artificial Kidney “WAK”) has been under development for some time in order to provide flexibility for dialysis patients. One of the most challenging aspects in the development of a portable dialysis device is in the efficient removal of urea, whose removal rate can be used to assess the efficiency of dialysis treatment. Urea removal can also reflect efficient removal of small ionic species such potassium as well.

In chronic kidney disease (“CKD”) patients, pre-dialysis serum urea levels are significantly higher than the normal range rising to between 20 and 40 mg/dL, while the urea concentration in dialysate is around 30 mg/dL. Miniaturization of the presently employed hemodialysis systems into highly effective, portable and low-cost devices (i.e., a Wearable Artificial Kidney “WAK”) has been under development for some time based on peritoneal dialysis (PD), hemofiltration (HF) and hemodialysis (HD). Again, one of the most challenging aspects in the development of a WAK is in the efficient removal of urea.

However, in WAK based on PD, urea is cleansed enzymatically using the enzyme urease, which finally converts urea into toxic ammonia and carbon dioxide. Thus, the sorbent system for PD must contain zirconium phosphate to remove ammonia, but zirconium phosphate also adsorbs potassium, calcium, magnesium and other cations and metals, and in doing so removes some necessary electrolytes also releasing undesirable hydrogen ions for which then another sorbent is needed. Zirconium carbonate is a kind of adsorbent which can adsorb hydrogen ions, but the adsorption is by ion exchange, so it releases bicarbonate, acetate, and to a lesser extent sodium; for HF to provide effective clearance, large ultrafiltration volumes with the corresponding return of large volumes of a replacement fluid are required. For a WAK based on HD to provide effective clearance, the sorbents used are in effect ion exchangers, that release bicarbonate and sodium. Moreover, the main disadvantage of a wearable HD device is that there is a risk of clotting in the extracorporeal circuit. These concerns about the disadvantages of recently-developed WAK systems, make the discovery of an adsorbent that adsorbs urea without causing other side effects desirable.

Additionally, a variety of medical condition treatments, including liver diseases, renal disease, hereditary urea cycle abnormalities, heart failure and dietary problems, require selective adsorption of small molecules, such as urea, from blood or blood plasma.

Carbon sorbents that are conventionally used for detoxification, cannot efficiently adsorb urea. Currently no materials capable of effectively adsorbing urea are available.

The present invention is directed to addressing at least some of these deficiencies.

### SUMMARY

MX-enes are a new class of two-dimensional (2D) transition metal carbides and nitrides that were discovered at Drexel University in 2011. Over the past few years, the range of materials contained within this family has expanded to include more than about 20 different MXenes. To date, most of the applications of MXenes focus on energy storage systems and their catalytic properties due because of their rich surface chemistries and high electronic conductivities. However, MXenes offer a large surface area and superior adsorptive potential for heavy metal ions and other materials.

Embodiments of the present invention(s) include methods of removing urea from solutions, each method comprising removing urea from an initial aqueous solution of urea, the method comprising subjecting the aqueous solution of urea to a MXene composition, at ambient or near ambient temperatures and conditions, so that urea is reduced from an initial concentration in the initial solution to a final concentration in a final solution. In some embodiments, the initial concentration of urea in the initial aqueous solution is in a range of from 10 mmol/L to 1000 mmol/L, or is initially in a concentration range from 10 mg/dL to 100 mg/dL, or around 30 mg/dL, and the final concentration is at least 10% less than the initial concentration.

In certain of further embodiments, the MXene composition is described as a composition comprising at least one layer having first and second surfaces, each layer described by a formula Mₙ₊₁Xₙ Tₓ and comprising:
a substantially two-dimensional array of crystal cells,
each crystal cell having an empirical formula of Mₙ₊₁Xₙ, such that each X is positioned within an octahedral array of M,
wherein M is at least one Group IIIB, IVB, VB, or VIB metal,
wherein each X is C, N, or a combination thereof,
n=1, 2, or 3; and wherein
Tₓ represents surface termination groups.

In other of these embodiments, the MXene composition is described as a composition comprising at least one layer having first and second surfaces, each layer comprising:
a substantially two-dimensional array of crystal cells,
each crystal cell having an empirical formula of M′₂M″ₙXₙ₊₁, such that each X is positioned within an octahedral array of M′ and M″, and where M″ₙ are present as individual two-dimensional array of atoms intercalated (sandwiched) between a pair of two-dimensional arrays of M′ atoms,
wherein M′ and M″ are different Group IIIB, IVB, VB, or VIB metals (especially where M′ and M″ are Ti, V, Nb, Ta, Cr, Mo, or a combination thereof),
wherein each X is C, N, or a combination thereof, and
n=1 or 2.

Variations of these MXene compositions and structures are provided herein.

The methods described herein are selective for adsorbing urea, and other defined small molecules and ions, and in various aspects, the aqueous solution further comprises amino acids, polypeptides, or blood plasma proteins, or one or more types of blood cells such as erythrocytes (red blood cells, RBCs), leukocytes (white blood cells), or thrombocytes (platelets), or one or more of such substances as glucose, fatty acids, and lactic acid. The aqueous solution may consist of or comprise blood or a blood product (e.g., blood serum, dialysate), and the ambient or near ambient temperatures and conditions used do not compromise the utility of the blood or blood product for later use by a human patient.

The methods are suitable for use in dialyses equipment, including portable dialysis equipment, both in through-pass and recycle modes.

Additional embodiments include those devices useful for operating the inventive methods, including those devices for removing urea from an aqueous solution of urea, the device comprising an exchangeable cartridge of MX-ene composition through which the solution is directed to pass, the passage adapted to allow the urea solution to contact the MX-ene composition contained in the cartridge.

### BRIEF DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific methods, devices, and systems disclosed. In addition, the drawings are not necessarily drawn to scale. In the drawings:

FIGS. 1A-D show a comparison of urea uptake capacities from aqueous solution.

FIG. 1A shows a comparison to control of the changes in the urea weight upon adsorption using two-dimensional (2D) titanium carbide MXene (Ti₃C₂Tₓ) as adsorbent with different mass-loadings (5.000, 2.500, 1.250, 0.625, 0.312 and 0.155 g−adsorbent dosage). FIG. 1B shows the removal efficiency in % using Ti₃C₂Tₓ as adsorbent with different mass-loadings (5.000, 2.500, 1.250, 0.625, 0.312 and 0.155 g−adsorbent dosage). FIG. 1C shows the amount of urea adsorbed during the adsorption time using 2D titanium carbide (Ti₃C₂Tₓ circles and Ti₂CTₓ, triangles) and molybdenum titanium carbide (Mo₂TiC₂Tₓ, squares) MXene (mass-loading was 0.155 g). FIG. 1D shows a comparison to control of the changes in the urea weight upon adsorption using 2D titanium carbide (Ti₃C₂Tₓ and Ti₂CTₓ) and molybdenum titanium carbide (Mo₂TiC₂Tₓ) (mass-loading was 0.155 g and adsorption time was 4 minutes). The volume of liquid phase containing urea for the adsorption was 6 mL with the initial concentration ~30 mg/dL.

FIGS. 2A-D show the adsorption of urea from dialysate (in 6 mL; initial concentration ~30 mg/dL). FIG. 2A shows changes in the urea concentration upon adsorption using two-dimensional (2D) titanium carbide MXene (Ti₃C₂Tₓ) as adsorbent with different mass-loadings (5.000, 2.500, 1.250, 0.625, 0.312 and 0.155 g−adsorbent dosage). FIG. 2B shows removal efficiency in % using Ti₃C₂Tₓ as adsorbent with different mass-loadings (5.000, 2.500, 1.250, 0.625, 0.312 and 0.155 g−adsorbent dosage). FIG. 2C shows the amount of urea adsorbed during the adsorption time using 2D titanium carbide (Ti₃C₂Tₓ, squares and Ti₂CTₓ, circles) and molybdenum titanium carbide (Mo₂TiC₂Tₓ, triangles) MXene (mass-loading was 0.155 g). FIG. 2D shows a comparison of the changes in the urea concentration upon adsorption using 2D titanium carbide (Ti₃C₂Tₓ and Ti₂CTₓ) and molybdenum titanium carbide (Mo₂TiC₂Tₓ) (mass-loading was 0.625 g and adsorption time was 4 minutes). The volume of dialysate for the adsorption was 6 mL with the initial concentration ~30 mg/dL.

FIGS. 3A-B shows a schematic representation of computation about interaction between urea and MXenes. FIG. 3A shows the most stable adsorption configurations and binding energies for each orientation of urea on the surfaces of MXenes. FIG. 3B shows the difference of charge density for parallel urea on the surfaces of MXenes; The turquoise and yellow regions indicate depletion and accumulation of electrons, respectively.

FIGS. 4A-B shows computation distances between MXenes layers. FIG. 4A shows configurations and distances between two layers of MXenes before and after intercalation of urea.

FIG. 4B shows interactions between MXene surface and protonated urea

FIGS. 5A-D ¹H NMR spectra. FIG. 5A shows an ¹H NMR spectrum of an aqueous urea solution 30 mg/dL. FIG. 5B shows an ¹H NMR spectrum after adsorption of urea from aqueous solution using Ti₃C₂Tₓ (mass loading 0.625 g; concentration of urea 30 mg/dL; volume 6 mL). FIG. 5C shows an ¹H NMR spectrum of an aqueous urea solution 3000 mg/dL; FIG. 5D shows an ¹H NMR spectrum after adsorption of urea from aqueous solution using Ti₃C₂Tₓ (mass loading 0.625 g; concentration of urea 3000 mg/dL; volume 6 mL).

FIG. 6 shows adsorption isotherms of urea on Ti₃C₂Tₓ (fit by Freundlich (solid blue line), Langmuir (solid red line) and Langmuir-Freundlich (solid light-blue line) models. Amount of urea adsorbed in mg per gram of adsorbent versus equilibrium concentration of urea using two-dimensional (2D) titanium carbide, Ti₃C₂Tₓ, as adsorbent (experimental points are marked with filled blue circles). The experimental adsorption data were fitted by Freundlich (solid light-blue line) for non-ideal adsorption that involves heterogeneous sorption with a non-uniform population of adsorption sites, Langmuir (solid brown line) for ideal adsorption, there are a fixed number of sites with the same adsorption energy available on the adsorbent surface and Langmuir-Freundlich (solid pink line) is a combination of the Langmuir and Freundlich isotherms.

FIG. 7 FIG. 7 shows cell viability measurement on direct contact of murine 3T3 fibroblast with Ti₃C₂Tₓ indicating no significant reduction in cell viability following exposure to increasing concentrations of Ti₃C₂Tₓ in contrast to graphene nanoparticles (GNP), graphene oxide (GO), and GO-Ag where a significant reduction occurred at the higher concentrations (p<0.001) (n=3, mean+/−sem).

FIGS. 8A-B shows overlay confocal images combining light, red and green fluorescent micrographs to show nanoparticles (NPs) and live/dead cell staining with calcein-AM (1p M) and ethidium homodimer (0.5 μM) in the no NPs control cells (FIG. 8A) and in MXene exposed cells at a NP concentration of 6.25 mg mL⁻¹ (FIG. 8B). The black NPs are clearly visible in the light microscopy overlay whilst the green fluorescence indicates a metabolizing cell fraction under the NP layer in both b and c. No red fluorescence indicating cell death was observed in the NP images.

### DETAILED DESCRIPTION OF ILLUSTRATIVE EMBODIMENTS

The present invention is directed to methods for polishing low levels of urea from aqueous solutions, including blood and blood products, dialysate from dialysis devices, and devices which may be used to employ these methods. Some of these embodiments are described in the appended claims. Contacting aqueous solutions of urea with MX-ene materials has been disclosed in one or more U.S. and international patent applications, for example, U.S. patent application Ser. No. 14/094,966 (“the '966 application,” filed Dec. 13, 2013), now U.S. Pat. No. 9,193,585. In contrast with the present disclosure, the aim in these previous disclosures was to prepare the resulting intercalated product in which the urea intercalated into the MX-ene composition, and the conditions were considerably more forcing than described herein. For example, in the '966 application, the conditions used to prepare the urea-intercalated MX-ene were described as contacting 5 mL of 50 wt. % aqueous solution of urea with 0.3 g of Ti₃C₂Tₓ and stirred for 24 h at 60 °C. In the instant disclosure, the removal of the urea from the aqueous solutions is better characterized as a polishing step, to remove low levels of urea from aqueous solutions (including blood and blood products) at ambient or near ambient temperatures (e.g., at or about 25 °C) over the course of minutes (e.g., in some cases, less than 5 minutes). At the levels described herein, urea is initially present at concentrations less than 1 mol/L. At these levels (1 mol/L of urea in water is equivalent to 60 g urea/L solution or about 6 wt %) the urea is present initially at levels which are more than an order of magnitude lower than the forcing conditions previously described. In other embodiments disclosed herein, urea can be removed from both aqueous urea solutions and dialysate directly from the uremic patients, wherein the initial urea concentration is about 30 mg/dL or 0.3 g urea/L solution, or more than 3 orders of magnitude lower than the forcing conditions described above. The results indicated that the adsorption can reach the equilibrium in less than 5 minutes. Nothing in the previous disclosures even suggests that MX-enes would be able to remove urea from solution with the surprising efficiency of kinetics and equilibrium constants necessary to affect the removals described herein. In particular, the contrast of the conditions previously used for urea with those used to intercalate other materials compounds further suggests that MXenes would require extremely forcing conditions to adsorb urea. Further, nothing in the previous references suggests any benefit of reacting MX-ene with urea solutions of such low concentrations or mild conditions. The methods described in this present disclosure are both remarkable and surprising in the face of the previous disclosures.

Embodiments of the present invention(s) include methods of removing urea from solutions, each method comprising subjecting the aqueous solution of urea to a MXene composition, at ambient or near ambient temperatures, so as to remove urea even from low level urea solutions, in effect “polishing” these aqueous solutions so as to reduce the concentration even further. In some embodiments, the urea is reduced from an initial concentration in the initial solution to a final concentration in a final solution, wherein the initial concentration of urea in the initial aqueous solution is in a range of from 10 to 20 mmol/L, from 20 to 40 mmol/L, from 40 to 80 mmol/L, from 80 to 160 mmol/L, from 160 to 200 mmol/L, from 200 to 400 mmol/L, from 400 to 600 mmol/L, from 600 to 800 mmol/L, from 800 to 1000 mmol/L, or in a range that is defined by two or more of these ranges, or is initially in a concentration range from 5 to 10 mg/dL, from 10 to 15 mg/dL, from 15 to 20 mg/dL, from 20 to 25 mg/dL, from 25 to 30 mg/dL, from 30 to 35 mg/dL, from 35 to 40 mg/dL, from 40 to 45 mg/dL, from 45 to 50 mg/dL, from 50 to 55 mg/dL, from 55 to 60 mg/dL, from 60 to 70 mg/dL, from 70 to 80 mg/dL, from 80 to 90 mg/dL, from 90 to 100 mg/dL, or in a range that is defined by two or more of these ranges, and the final concentration is at least 10%, 20%, 30%, 40%, 50%, 60%, 70%, 80%, or 90% less than the initial concentration. In independent embodiments, the final concentration of the urea in the final solution is less than 100 mmol/L, less than 80 mmol/L, less than 60 mmol/L, less than 40 mmol/L, less than 20 mmol/L, less than 10 mmol/L, less than 5 mmol/L, or less than 1 mmol/L, or any combination of two or more of these ranges. In some cases, the levels of urea may even be undetectable by standard analytical methods. Specific exemplary ranges and levels, both initial and final, are described in the Examples.

It is convenient to measure these levels by any number of quantitative or semi-quantitative analytical methods, though ¹H-NMR and liquid chromatography, including high performance liquid chromatography (HPLC) and other chemical reaction coupled colorimetric and fluorometric methods, have been shown to be especially useful in this regard. Commercial Urea Assay Kits may also be used to measure the concentrations of urea.

These MXene compositions described herein are also sometimes described in terms of the phrase “MX-enes” or “MX-ene compositions.” MXenes may be described as two-dimensional transition metal carbides, nitrides, or carbonitrides comprising at least one layer having first and second surfaces, each layer described by a formula Mₙ₊₁Xₙ Tₓ and comprising:
a substantially two-dimensional array of crystal cells,
each crystal cell having an empirical formula of Mₙ₊₁Xₙ, such that each X is positioned within an octahedral array of M,
wherein M is at least one Group IIIB, IVB, VB, or VIB metal,
wherein each X is C, N, or a combination thereof,
n=1, 2, or 3; and wherein
Tₓ represents surface termination groups.

These so-called MXene compositions have been described in U.S. Pat. No. 9,193,595 and Application PCT/US2015/051588, filed Sep. 23, 2015, each of which is incorporated by reference herein in its entirety at least for its teaching of these compositions, their (electrical) properties, and their methods of making. That is, any such composition described in this application is considered as applicable for use in the present methods and within the scope of the present invention. For the sake of completeness, M can be at least one of Sc, Y, Lu, Ti, Zr, Hf, V, Nb, Ta, Cr, Mo, or W. In certain embodiments in this class, M is at least one Group IVB, Group VB, or Group VIB metal, preferably Ti, Mo, Nb, V, or Ta. Certain of these compositions include those having one or more empirical formula wherein Mₙ₊₁Xₙ comprises Sc₂C, Ti₂C, V₂C, Cr₂C, Cr₂N, Zr₂C, Nb₂C, Hf₂C, Ti₃C₂, V₃C₂, Ta₃C₂, Ti₄C₃, V₄C₃, Ta₄C₃, Sc₂N, Ti₂N, V₂N, Cr₂N, Cr₂N, Zr₂N, Nb₂N, Hf₂C, Ti₃N₂, V₃C₂, Ta₃C₂, Ti₄N₃, V₄C₃, Ta₄N₃ or a combination or mixture thereof. In particular embodiments, the Mₙ₊₁Xₙ structure comprises Ti₃C₂, Ti₂C, Ta₄C₃ or (V_{1/2}Cr_{1/2})₃C₃. In some embodiments, M is Ti or Ta, and n is 1, 2, or 3, for example having an empirical formula Ti₃C₂ or Ti₂C. In some of these embodiments, at least one of said surfaces of each layer has surface terminations comprising hydroxide, oxide, sub-oxide, or a combination thereof. In certain preferred embodiments, the MXene composition is described by a formula Mₙ₊₁Xₙ Tₓ, where Mₙ₊₁Xₙ are Ti₂CTₓ, Mo₂TiC₂Tₓ, Ti₃C₂Tₓ, or a combination thereof, and Tₓ is as described herein. Those embodiments wherein M is Ti, and n is 1 or 2, preferably 2, are especially preferred.

In other embodiments, the methods use compositions, wherein the two-dimensional transition metal carbide, nitrides, or carbonitride comprises a composition having at least one layer having first and second surfaces, each layer comprising:
a substantially two-dimensional array of crystal cells,
each crystal cell having an empirical formula of M′₂M″ₙ~Xₙ₊₁, such that each X is positioned within an octahedral array of M′ and M″, and where M″ₙ are present as individual two-dimensional array of atoms intercalated (sandwiched) between a pair of two-dimensional arrays of M′ atoms,
wherein M′ and M″ are different Group IIIB, IVB, VB, or VIB metals (especially where M′ and M″ are Ti, V, Nb, Ta, Cr, Mo, or a combination thereof),
wherein each X is C, N, or a combination thereof, preferably C; and
n=1 or 2.

These compositions are described in greater detail in Application PCT/US2016/028354, filed Apr. 20, 2016, which is incorporated by reference herein in its entirety at least for its teaching of these compositions and their methods of making. For the sake of completeness, in some embodiments, M′ is Mo, and M″ is Nb, Ta, Ti, or V, or a combination thereof. In other embodiments, n is 2, M′ is Mo, Ti, V, or a combination thereof, and M″ is Cr, Nb, Ta, Ti, or V, or a combination thereof. In still further embodiments, the empirical formula M′₂M″Xₙ₊₁ comprises Mo₂TiC₂, Mo₂VC₂, Mo₂TaC₂, Mo₂NbC₂, Mo₂Ti₂C₃, Cr₂TiC₂, Cr₂VC₂, Cr₂TaC₂, Cr₂NbC₂, Ti₂NbC₂, Ti₂TaC₂, V₂TaC₂, or V₂TiC₂, preferably Mo₂TiC₂, Mo₂VC₂, Mo₂TaC₂, or Mo₂NbC₂, or their nitride or carbonitride analogs. In still other embodiments, M′₂M″ₙXₙ₊₁ comprises Mo₂Ti₂C₃, Mo₂V₂C₃, Mo₂Nb₂C₃, Mo₂Ta₂C₃, Cr₂Ti₂C₃, Cr₂V₂C₃, Cr₂Nb₂C₃, Cr₂Ta₂C₃, Nb₂Ta₂C₃, Ti₂Nb₂C₃, Ti₂Ta₂C₃, V₂Ta₂C₃, V₂Nb₂C₃, or V₂Ti₂C₃, preferably Mo₂Ti₂C₃, Mo₂V₂C₃, Mo₂Nb₂C₃, Mo₂Ta₂C₃, Ti₂Nb₂C₃, Ti₂Ta₂C₃, or V₂Ta₂C₃, or their nitride or carbonitride analogs.

Each of these compositions having empirical crystalline formulae Mₙ₊₁Xₙ or M′₂M″ₙXₙ₊₁ are described in terms of comprising at least one layer having first and second surfaces, each layer comprising a substantially two-dimensional array of crystal cells. In some embodiments, these compositions comprise layers of individual two-dimensional cells. In other embodiments, the compositions comprise a plurality of stacked layers. Additionally, in some embodiments, at least one of said surfaces of each layer has surface terminations (optionally designated “Tₛ” or “Tₓ”) comprising alkoxide, carboxylate, halide, hydroxide, hydride, oxide, sub-oxide, nitride, sub-nitride, sulfide, thiol, or a combination thereof. In some embodiments, at least one of said surfaces of each layer has surface terminations comprising alkoxide, fluoride, hydroxide, oxide, sub-oxide, or a combination thereof. In still other embodiments, both surfaces of each layer have said surface terminations comprising alkoxide, fluoride, hydroxide, oxide, sub-oxide, or a combination thereof. As used herein the terms “sub-oxide,” “sub-nitride,” or “sub-sulfide” is intended to connote a composition containing an amount reflecting a sub-stoichiometric or a mixed oxidation state of the M metal at the surface of oxide, nitride, or sulfide, respectively. For example, various forms of titania are known to exist as TiOₓ, where x can be less than 2. Accordingly, the surfaces of the present invention may also contain oxides, nitrides, or sulfides in similar sub-stoichiometric or mixed oxidation state amounts.

In the methods, these MXenes may comprise simple individual layers, a plurality of stacked layers, or a combination thereof. Each layer may independently comprise surfaces functionalized by any of the surface coating features described herein (e.g., as in alkoxide, carboxylate, halide, hydroxide, hydride, oxide, sub-oxide, nitride, sub-nitride, sulfide, thiol, or a combination thereof) or may be also partially or completely functionalized by polymers, either on the surface of individual layers, for example, where the two-dimensional compositions are embedded within a polymer matrix, or the polymers may be intercalated between layers to form structural composites, or both.

The methods may be applied to aqueous solutions, including physiologically important aqueous fluids, with or without the need to remove extraneous (e.g., non-urea) materials. In certain embodiments, in addition to the urea, the aqueous solution further comprises amino acids, polypeptides, or blood plasma proteins. Further, the aqueous solution may further comprise one or more of types of blood cells such as erythrocytes (red blood cells, RBCs), leukocytes (white blood cells), or thrombocytes (platelets). Still further, the aqueous solution may also comprise one or more of substances such as glucose, fatty acids, and lactic acid.

The methods are useful in a dialysis context—i.e., where the aqueous solution is or comprises blood or a blood product (e.g., blood serum, dialysate). That is, the methods are operable at ambient or near ambient temperatures and under conditions suitable so as not to compromise the utility of the blood or blood product for later use by a human patient. In these aspects, the initial urea concentrations are physiologically relevant to a human patient.

In some aspects of the present invention(s), the methods comprise contacting the aqueous urea solutions with fresh or previously used MXene compositions. Likewise, the aqueous solutions may be contacted with one or more fresh batches of MXene materials, for example, where the final aqueous solution of urea of a first pass cleaning is contacted with previously unexposed (i.e., fresh) MXene compositions.

“Contacting” may comprise adding MXene compositions into a quantity of the aqueous solution comprising urea, followed by separating the urea-adsorbed-MXene from the bulk solution, for example by filtration. Or, the contacting may comprise passing the aqueous urea solution though a bed or beds or across a surface comprising one of more MXene compositions. Or the contacting may comprise a method involving both bulk and bed or surface processing.

Once the MXenes have adsorbed the urea and are separated from the aqueous solution, the MXenes may be replaced or re-generated by flushing with suitable solvents.

To this point, the description has been in terms of methods, but the invention(s) also include those devices the useful for affecting these methods. That is, certain embodiments also include those devices for removing urea from an aqueous solution of urea, the device comprising an exchangeable cartridge of MX-ene composition through or along which the solution is directed to pass, the passage being adapted to allow the urea solution to contact the MX-ene composition contained in the cartridge. These cartridges also represent embodiments of this invention, and may be configured to provide for the percolation of the aqueous solutions through bulk quantities of the MXenes, or may be configured with MXene-coated channels, along which the solutions are directed to pass. Within these devices or cartridges, the MXene composition is or comprises any one or more of the MXene compositions described herein.

### TERMS

In the present disclosure the singular forms “a,” “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art.

When a value is expressed as an approximation by use of the descriptor “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about.” In other cases, the gradations used in a series of values may be used to determine the intended range available to the term “about” for each value. Where present, all ranges are inclusive and combinable. That is, references to values stated in ranges include every value within that range.

It is to be appreciated that certain features of the invention which are, for clarity, described herein in the context of separate embodiments, may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, while an embodiment may be described as part of a series of steps or part of a more general structure, each said step may also be considered an independent embodiment in itself, combinable with others.

The transitional terms “comprising,” “consisting essentially of,” and “consisting” are intended to connote their generally in accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps and those that do not materially affect the basic and novel characteristic(s) of the claimed invention. Embodiments described in terms of the phrase “comprising” (or its equivalents), also provide, as embodiments, those which are independently described in terms of “consisting of” and “consisting essentially of” For those composition embodiments provided in terms of “consisting essentially of,” the basic and novel characteristic(s) is the ability to remove low levels of urea from aqueous solutions in a timely manner as described herein or as explicitly specified.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

Throughout this specification, words are to be afforded their normal meaning, as would be understood by those skilled in the relevant art. However, so as to avoid misunderstanding, the meanings of certain terms will be specifically defined or clarified.

The terms “MXenes” or “two-dimensional (2D) crystalline transition metal carbides” or two-dimensional (2D) transition metal carbides” may be used interchangeably to refer collectively to compositions described herein as comprising substantially two-dimensional crystal lattices of the general formulae Mₙ₊₁Xₙ (Tₛ), M₂A₂X(Tₛ). and M′₂M″ₙXₙ₊₁(Tₛ), where M, M′, M″, A, X, and Tₛ are defined herein. Supplementing the descriptions herein, Mₙ₊₁Xₙ (Tₛ) (including M′₂M″ₘXₘ₊₁(Tₛ) compositions) may be viewed as comprising free standing and stacked assemblies of two dimensional crystalline solids. Collectively, such compositions are referred to herein as “Mₙ₊₁Xₙ (Tₛ),” “MXene,” “MXene compositions,” or “MXene materials.” Additionally, these terms “Mₙ₊₁Xₙ (Tₛ),” “MXene,” “MXene compositions,” or “MXene materials” can also independently refer to those compositions derived by the chemical exfoliation of MAX phase materials, whether these compositions are present as free-standing 2-dimensional or stacked assemblies (as described further below). These compositions may be comprised of individual or a plurality of such layers. In some embodiments, the MXenes comprising stacked assemblies may be capable of, or have atoms, ions, or molecules, that are intercalated between at least some of the layers. In other embodiments, these atoms or ions are lithium.

The term “crystalline compositions comprising at least one layer having first and second surfaces, each layer comprising a substantially two-dimensional array of crystal cells” refers to the unique character of these materials. For purposes of visualization, the two-dimensional array of crystal cells may be viewed as an array of cells extending in an x-y plane, with the z-axis defining the thickness of the composition, without any restrictions as to the absolute orientation of that plane or axes. It is preferred that the at least one layer having first and second surfaces contain but a single two-dimensional array of crystal cells (that is, the z-dimension is defined by the dimension of approximately one crystal cell), such that the planar surfaces of said cell array defines the surface of the layer; it should be appreciated that real compositions may contain portions having more than single crystal cell thicknesses.

That is, as used herein, “a substantially two-dimensional array of crystal cells” refers to an array which preferably includes a lateral (in x-y dimension) array of crystals having a thickness of a single unit cell, such that the top and bottom surfaces of the array are available for chemical modification.

The following listing of Embodiments is intended to complement, rather than displace or supersede, the previous descriptions.

Embodiment 1. A method of removing urea from an initial aqueous solution of urea, the method comprising subjecting the aqueous solution of urea to a MXene composition, at ambient or near ambient temperatures and conditions, so that the urea is reduced from an initial concentration in the initial solution to a final concentration in a final solution, wherein the initial concentration of urea in the initial aqueous solution is in a range of from 10 to 20 mmol/L, from 20 to 40 mmol/L, from 40 to 80 mmol/L, from 80 to 160 mmol/L, from 160 to 200 mmol/L, from 200 to 400 mmol/L, from 400 to 600 mmol/L, from 600 to 800 mmol/L, from 800 to 1000 mmol/L, or is defined by two or more of these ranges, or is initially in a concentration range from 5 to 10 mg/dL, from 10 to 15 mg/dL, from 15 to 20 mg/dL, from 20 to 25 mg/dL, from 25 to 30 mg/dL, from 30 to 35 mg/dL, from 35 to 40 mg/dL, from 40 to 45 mg/dL, from 45 to 50 mg/dL, from 50 to 55 mg/dL, from 55 to 60 mg/dL, from 60 to 70 mg/dL, from 70 to 80 mg/dL, from 80 to 90 mg/dL, from 90 to 100 mg/dL, or is defined by two or more of these ranges, and the final concentration is at least 10%, 20%, 30%, 40%, 50%, 60%, 70%, 80%, or 90% less than the initial concentration.

Embodiment 2. The method of Embodiment 1, wherein the MXene composition comprises a composition comprising at least one layer having first and second surfaces, each layer described by a formula Mₙ₊₁Xₙ Tₓ and comprising:
a substantially two-dimensional array of crystal cells,
each crystal cell having an empirical formula of Mₙ₊₁Xₙ, such that each X is positioned within an octahedral array of M,
wherein M is at least one Group IIIB, IVB, VB, or VIB metal,
wherein each X is C, N, or a combination thereof,
n=1, 2, or 3; and wherein
Tₓ represents surface termination groups.

Embodiment 3. The method of Embodiment 2, wherein the MXene composition comprises a plurality of stacked layers

Embodiment 4. The method of Embodiment 2 or 3, wherein at least one of said surfaces of each layer has surface termination groups (Tₓ) comprising alkoxide, carboxylate, halide, hydroxide, hydride, oxide, sub-oxide, nitride, sub-nitride, sulfide, thiol, or a combination thereof

Embodiment 5. The method of any one of Embodiments 2 to 4, wherein at least one of said surfaces of each layer has surface terminations comprising alkoxide, fluoride, hydroxide, oxide, sub-oxide, or a combination thereof

Embodiment 6. The method of any one of Embodiments 2 to 5, wherein both surfaces of each layer have said surface terminations comprising alkoxide, fluoride, hydroxide, oxide, sub-oxide, or a combination thereof.

Embodiment 7. The method of any one of Embodiments 2 to 6, wherein M is at least one Group IVB, Group VB, or Group VIB metal, preferably Ti, Mo, Nb, V, or Ta.

Embodiment 8. The method of any one of Embodiments 1 to 7, wherein the MXene composition is described by a formula Mₙ₊₁Xₙ Tₓ, where Mₙ₊₁Xₙ are Ti₂CTₓ, Mo₂TiC₂Tₓ, Ti₃C₂Tₓ, or a combination thereof, and Tₓ is as described herein.

Embodiment 9. The method of any one of Embodiments 2 to 7, wherein M is Ti, and n is 1 or 2, preferably 2.

Embodiment 10. The method of Embodiment 1, wherein the MXene composition comprises a composition comprising at least one layer having first and second surfaces, each layer comprising:
a substantially two-dimensional array of crystal cells,
each crystal cell having an empirical formula of M′₂M″ₙXₙ₊₁, such that each X is positioned within an octahedral array of M′ and M″, and where M″ₙ are present as individual two-dimensional array of atoms intercalated (sandwiched) between a pair of two-dimensional arrays of M′ atoms,
wherein M′ and M″ are different Group IIIB, IVB, VB, or VIB metals (especially where M′ and M″ are Ti, V, Nb, Ta, Cr, Mo, or a combination thereof),
wherein each X is C, N, or a combination thereof, and
n=1 or 2.

Embodiment 11. The method of Embodiment 10, wherein n is 1, M′ is Mo, and M″ is Nb, Ta, Ti, or V, or a combination thereof.

Embodiment 12. The method of Embodiment 10 or 11, wherein n is 2, M′ is Mo, Ti, V, or a combination thereof, and M″ is Cr, Nb, Ta, Ti, or V, or a combination thereof.

Embodiment 13. The method of any one of Embodiments 10 to 12, wherein M′₂M″ₙXₙ₊₁ comprises Mo₂TiC₂, Mo₂VC₂, Mo₂TaC₂, Mo₂NbC₂, Mo₂Ti₂C₃, Cr₂TiC₂, Cr₂VC₂, Cr₂TaC₂, Cr₂NbC₂, Ti₂NbC₂, Ti₂TaC₂, V₂TaC₂, or V₂TiC₂, or a nitride or carbonitride analog thereof

Embodiment 14. The method of any one of Embodiments 10 to 13, wherein M′₂M″ₙXₙ₊₁ comprises Mo₂TiC₂, Mo₂VC₂, Mo₂TaC₂, or Mo₂NbC₂, or a nitride or carbonitride analog thereof.

Embodiment 15. The method of any one of Embodiments 10 to 14, wherein M′₂M″ₙXₙ₊₁ comprises Mo₂Ti₂C₃, Mo₂V₂C₃, Mo₂Nb₂C₃, Mo₂Ta₂C₃, Cr₂Ti₂C₃, Cr₂V₂C₃, Cr₂Nb₂C₃, Cr₂Ta₂C₃, Nb₂Ta₂C₃, Ti₂Nb₂C₃, Ti₂Ta₂C₃, V₂Ta₂C₃, V₂Nb₂C₃, or V₂Ti₂C₃, or a nitride or carbonitride analog thereof.

Embodiment 16. The method of any one of Embodiments 10 to 15, wherein M′₂M″ₙXₙ₊₁ comprises Mo₂Ti₂C₃, Mo₂V₂C₃, Mo₂Nb₂C₃, Mo₂Ta₂C₃, Ti₂Nb₂C₃, Ti₂Ta₂C₃, or V₂Ta₂C₃, or a nitride or carbonitride analog thereof.

Embodiment 17. The method of any one of Embodiments 10 to 16, wherein the MXene composition comprises a plurality of stacked layers

Embodiment 18. The method of any one of Embodiments 10 to 17, wherein at least one of said surfaces of each layer has surface terminations comprising alkoxide, carboxylate, halide, hydroxide, hydride, oxide, sub-oxide, nitride, sub-nitride, sulfide, thiol, or a combination thereof

Embodiment 19. The method of any one of Embodiments 10 to 18, wherein at least one of said surfaces of each layer has surface terminations comprising alkoxide, fluoride, hydroxide, oxide, sub-oxide, or a combination thereof

Embodiment 20. The method of any one of Embodiments 10 to 19, wherein both surfaces of each layer have said surface terminations comprising alkoxide, fluoride, hydroxide, oxide, sub-oxide, or a combination thereof.

Embodiment 21. The method of any one of Embodiments 1 to 20, wherein the MXene composition is any of the compositions described in any one of U.S. patent application Ser. No. 14/094,966 (filed Dec. 3, 2013), 62/055,155 (filed Sep. 25, 2014), 62/214,380 (filed Sep. 4, 2015), 62/149,890 (filed Apr. 20, 2015), 62/127,907 (filed Mar. 4, 2015) or International Applications PCT/US2012/043273 (filed Jun. 20, 2012), PCT/US2013/072733 (filed Dec. 3, 2013), PCT/US2015/051588 (filed Sep. 23, 2015), PCT/US2016/020216 (filed Mar. 1, 2016), or PCT/US2016/028,354 (filed Apr. 20, 2016), each of which is incorporated by reference at least for its teaching of the compositions and methods of making the same.

Embodiment 22. The method of any one of Embodiments 1 to 21, wherein the aqueous solution further comprises amino acids, polypeptides, or blood plasma proteins.

Embodiment 23. The method of any one of Embodiments 1 to 22, wherein the aqueous solution further comprises one or more of erythrocytes (red blood cells, RBCs), leukocytes (white blood cells), or thrombocytes (platelets).

Embodiment 24. The method of any one of Embodiments 1 to 23, wherein the aqueous solution further comprises one or more of such as glucose, fatty acids, and lactic acid.

Embodiment 25. The method of any one of Embodiments 1 to 24, wherein the aqueous solution is or comprises blood or a blood product (e.g., blood serum, dialysate), and the ambient or near ambient temperatures and conditions used do not compromise the utility of the blood or blood product for later use by a human patient.

Embodiment 26. The method of any one of Embodiments 1 to 25, wherein the initial concentration is a physiologically relevant concentration to a human patient.

Embodiment 27. The method of any one of Embodiments 1 to 26, wherein the final aqueous solution of urea is contacted with previously unexposed (i.e., fresh) MXene compositions.

Embodiment 28. The method of any one of Embodiments 1 to 27, final aqueous solution of urea is contacted with previously unexposed (i.e., fresh) MXene compositions more than once in a recycle scenario.

Embodiment 29. The method of any one of Embodiments 1 to 28, wherein the final concentration of the urea in the final solution is less than 100 mmol/L, less than 80 mmol/L, less than 60 mmol/L, less than 40 mmol/L, less than 20 mmol/L, less than 10 mmol/L, less than 5 mmol/L, or less than 1 mmol/L, when measured by ¹H NMR, the ¹H NMR method as being well understood by those skilled in the art, as well as those methods described as herein.

Embodiment 30. The method of any one of Embodiments 1 to 29, wherein the urea is undetectable in the final solution by ¹H NMR, the ¹H NMR method as being well understood by those skilled in the art, as well as those methods described as herein.

Embodiment 31. A device for removing urea from an aqueous solution of urea, the device comprising an exchangeable cartridge of MXene composition through which the solution is directed to pass, the passage adapted to allow the urea solution to contact the MXene composition contained in the cartridge. In certain of these Embodiments, the MXene composition is or comprises any one or more of the MXene compositions described herein.

Embodiment 32. The device of Embodiment 31, wherein the device is adapted to allow the aqueous solution of urea to percolate through at least a portion of the MXene composition.

Embodiment 33. The device of Embodiment 31 or 32, wherein the device is adapted to affect the method of any one of Embodiments 1 to 30.

### EXAMPLES

The following Examples are provided to illustrate some of the concepts described within this disclosure. While each Example is considered to provide specific individual embodiments of composition, methods of preparation and use, none of the Examples should be considered to limit the more general embodiments described herein. In particular, while the examples provided here focus on specific MXene materials, it is believed that the principles described are relevant to other such MXene materials. Accordingly, the descriptions provided here should not be construed to limit the disclosure, and the reader is advised to look to the nature of the claims as a broader description.

In the following examples, efforts have been made to ensure accuracy with respect to numbers used (e.g. amounts, temperature, etc.) but some experimental error and deviation should be accounted for. Unless indicated otherwise, temperature is in degrees C, pressure is at or near atmospheric.

Herein, we present the adsorption behavior of Ti₃C₂Tₓ both in the aqueous solution of urea and dialysate. First-principle calculations indicated urea that has stable adsorption on MXene surface groups in both parallel and vertical orientations. The results of the adsorption studies showed rapid urea removal at low concentrations in dialysate (~30 mg/dL). The adsorption results showed that the adsorption efficiency in water was higher than in dialysate due to the competition of other biomolecules present in the dialysate. The comparison of various MXenes showed that Ti₃C₂Tₓ had better adsorption performance in both in aqueous solutions and dialysate, compared to Ti₂CTₓ and Mo₂TiC₂Tₓ. Cytotoxicity assessment of MXene Ti₃C₂Tₓ showed that, at the tested concentrations, the MXene had no significant effect on cell viability over an incubation period of 24 hours, which meant the Ti₃C₂Tₓ has good biocompatibility and can be used in biomedical applications. Thus, Ti₃C₂Tₓ was a promising material for removal of urea from uremic patients.

### Example 1: Materials

Urea crystals were purchased from Sigma-Aldrich (99.9-101.0%, calc. on dry substance) and urea assay kit (DIUR-100) from BioAssay Systems. The dialysate samples were collected from the uremia patients (Cedars-Sinai Medical Center, Los Angeles, CA) and stored at −80 °C until used. The Ti₃C₂Tₓ MXene and its precursors Ti₃AlC₂, Mo₂TiC₂Tₓ and Ti₂CTₓ as well as oxidized Nanodiamond UD90 were synthesized. Ti₃C₂Tₓ was synthesized as described previously. Briefly, 5 g of Ti₃AlC₂ (<37 μm particle size) powder was added into hydrofluoric acid (HF, 10%, 50 mL) solution over 2 min. The solution was stirred for 24 h at 35 °C. After that, the multilayer Ti₃C₂Tₓ was obtained by washing with deionized (DI) water by centrifugation and decantation several times until the pH of suspension reached ~6. Similarly, the Mo₂TiC₂Tₓ and Ti₂CTₓ were synthesized by etching in 50% HF at 55 °C for 72 h and in 10% HF at 35 °C for 18 hr, respectively.

The urea characteristic signal in the aqueous solutions before and after adsorption were determined using ¹H-NMR (Varian Inova 500 NMR Spectrometer). The samples were prepared by adding 25 μL D₂O and 475 μL urea aqueous solution to a 5 mm NMR tube. CellTiter 96© Aqueous One Solution Cell Proliferation Assay (Promega Corporation Cat. #G3580) was used to study biocompatibility. Hydrochloric acid was purified by sterilized autoclaving in media, and Ag nanoparticles were purchased from Sigma-Aldrich. ATCC murine fibroblast cell line 3T3 grown in Dulbecco's Modified Eagle Medium supplemented with foetal bovine serum, live-dead stain (Molecular Probes)—calcein-acetoxymethyl ester (calcein-AM) and ethidium homodimer-1 (EthD-1) were used to study biocompatibility.

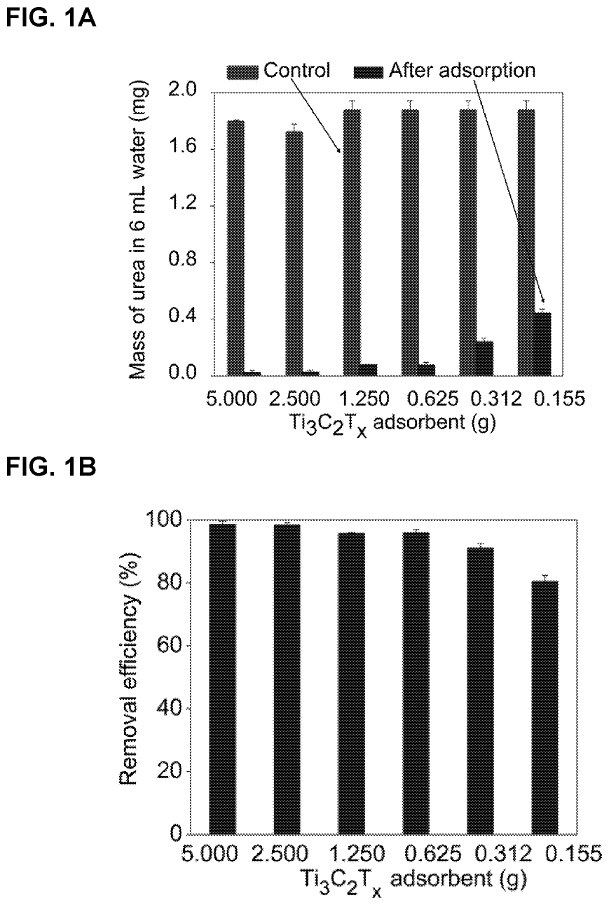

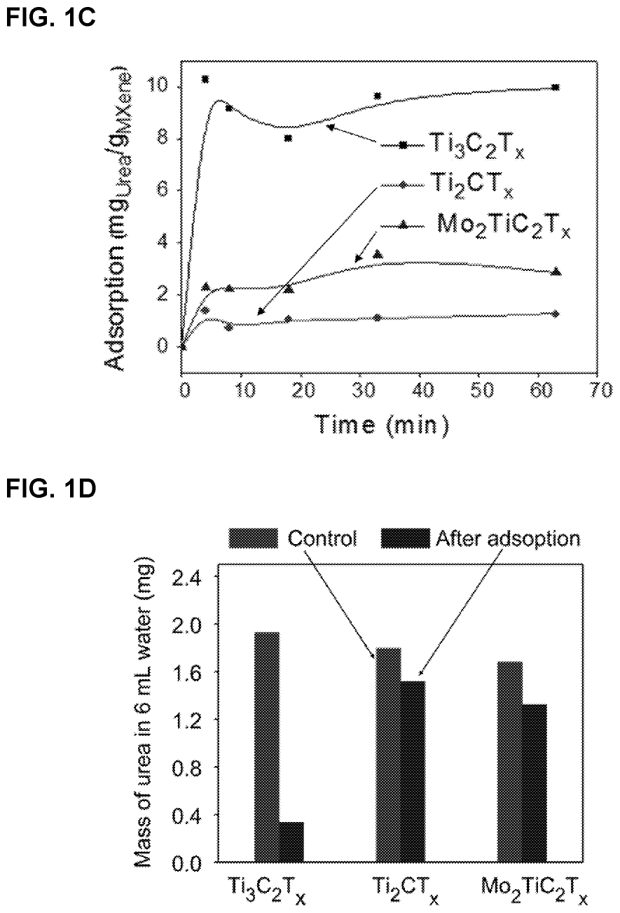

Example 1: In a first set of experiments, urea was dissolved in water to reach a 30 mg/dL urea concentration. Then different masses of MXene (Ti₃C₂Tₓ) powder (5.000, 2.500, 1.250, 0.625, 0.312 g and 0.155 g) were added into 6 mL of urea aqueous solution, mixed for 3 minutes by manual shaking and then kept on a test tube rack at room temperature. The concentration of urea was examined by reading the optical density at 520 nm using urea assay kit. At mass loadings of 5, 2.5 and 1.25 g, urea assay kit results indicated that when Ti₃C₂Tₓ MXene with masses of 5, 2.5, 1.25 and 0.625 g were used, the urea was almost completely adsorbed (>95% adsorption) after only 4 minutes of MXene being in the solution, 3 min shaking and 1 min sitting on the test tube rack (FIG. 1A). At lower mass loading of Ti₃C₂Tₓ powder, 0.312 and 0.155 g, urea adsorption was not as high as 95% (FIG. 1B). Similar urea adsorption was observed for all the reaction durations from 1 min to 1h, indicating that MXene effective reaction time is less than 5 minutes. We also investigated the adsorption kinetics of two other MXenes, Ti₂CTₓ and Mo₂TiC₂Tₓ and compared them with Ti₃C₂Tₓ adsorption. The results indicate that all materials with a mass-loading of 0.155 g adsorbed urea rapidly within 4 min, after which, adsorption showed a small change by increasing the contact time indicating that an equilibrium state was obtained (FIG. 1C). The results indicate that Ti₃C₂Tₓ shows the best adsorption performance with the highest urea removal efficiency from aqueous solution of these three MXenes (FIG. 1D)

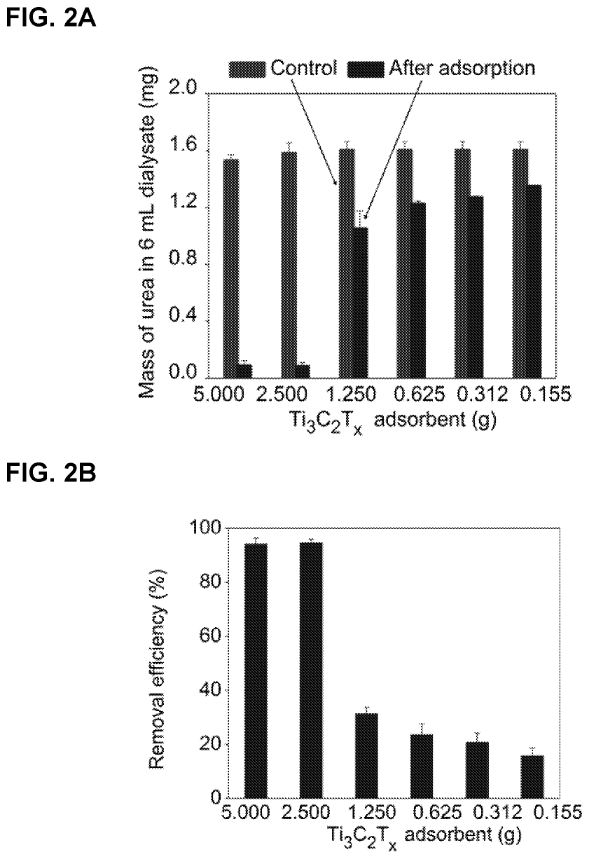

Example 2: In a second set of experiments, dialysate from uremia patients with 30 mg/dLurea concentration was used. Similar testing steps as for the Example 2 were used: different masses of MXene (Ti₃C₂Tₓ) powder (5.000, 2.500, 1.250, 0.625, 0.312, and 0.155 g) were added into 6 mL of the dialysate solution, mixed for 3 minutes by manual shaking and then kept on a test tube rack at room temperature. The concentration of urea was examined by reading the optical density at 520 nm. Urea adsorption was measured after addition of 5.000, 2.500, 1.250, 0.625, 0.312, 0.155 g (FIG. 2A), with the mass of Ti₃C₂Tₓ are 5.000 and 2.500 g of MXene, urea adsorption in dialysate can reach to 94%. However, when the mass of MXenes reduce under 1.250 g, the adsorption efficiency reduced dramatically to under 31% (FIG. 2B). The adsorption kinetics of two other MXenes, Ti₂CTₓ and Mo₂TiC₂Tₓ were also investigated and compared with Ti₃C₂Tₓ adsorption in dialysate. The results indicated that all materials with a mass-loading of 0.625 g adsorbed urea rapidly within 4 min, after which, adsorption showed a small change by increasing the contact time indicating that an equilibrium state was obtained (FIG. 2C). The results indicate that Ti₃C₂Tₓ showed the best adsorption performance with the highest urea removal efficiency from dialysate solution of these three MXenes (FIG. 2D).

These initial measurements showed that Ti₃C₂Tₓ MXene removed biologically relevant amounts of urea. It is reasonably expected that other MXenes can provide the same or maybe even more efficient adsorption. Such high adsorption ability has never been reported for any material, which makes MXene the best synthetic material to remove urea by adsorption. This process can replace the currently used dialysis procedure, allowing development of a wearable kidney for renal disease patients that currently require regular or constant dialysis treatment in a hospital.

### Example 3: Computational Details

To understand the interaction between urea and MXenes, first-principle calculations were performed to investigate the adsorption behaviors for urea on the MXene surface. Ti₃C₂Tₓ was chosen as the representation for the following discussions and three different surface terminations (—OH, —O or —F) are considered. The binding energy Eb of urea on MXene surface was defined as:

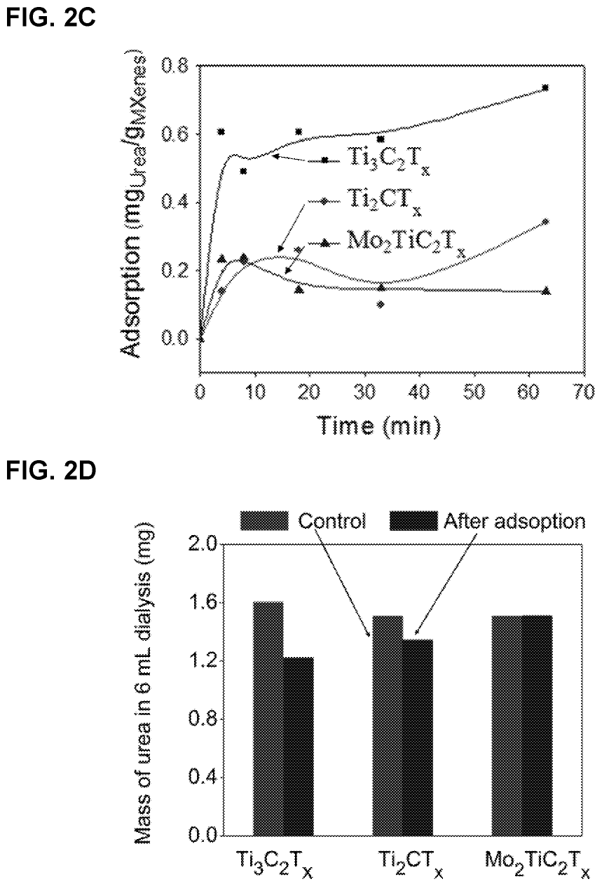

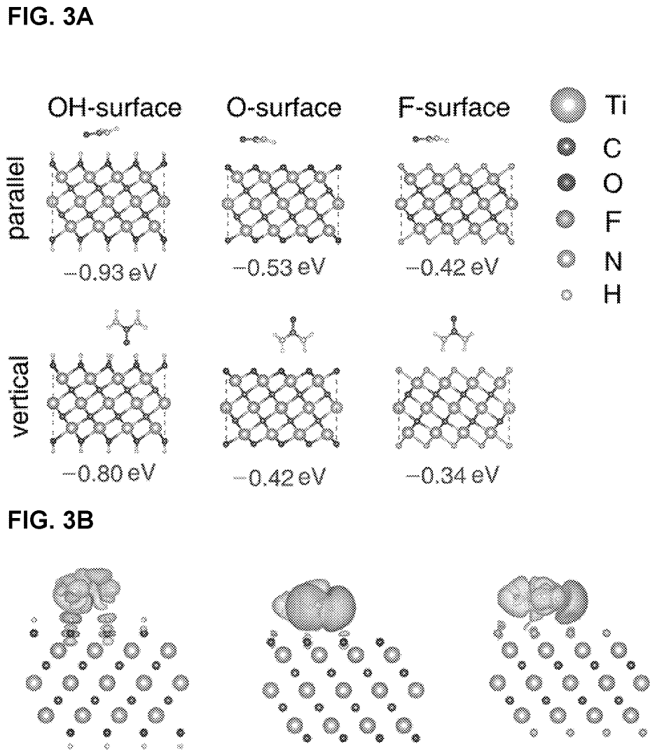

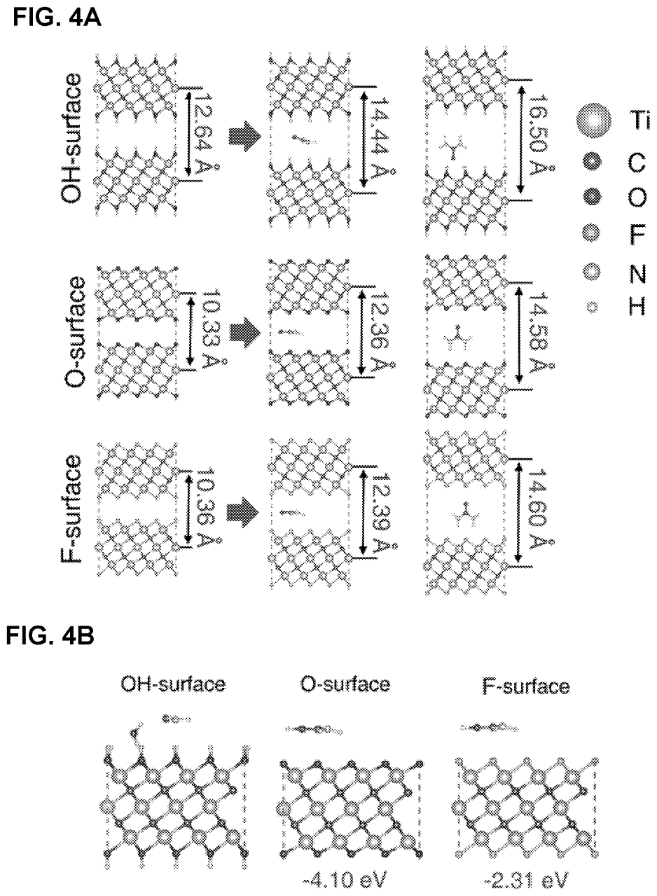

Eb=E_{MXenes+urea}−(E_{MXenes}+Eᵤᵣₑₐ),

where EM_{Xenes+urea} is the total energy of MXene with a urea molecular, E_{MXenes} is the total energy of MXene and Eᵤᵣₑₐ is the total energy of the urea molecular. The most stable adsorption configurations for each orientation (parallel or vertical) and their binding energies are exhibited in FIG. 3A. The binding energies on different surfaces range from −0.34 to −0.93 eV. The calculations showed that regardless of surface terminations (—OH, —O or —F), the urea molecule “preferred” the parallel adsorption configurations. Moreover, urea had the most stable adsorption on OH-surface with binding energy of −0.93 and −0.8 eV for parallel and vertical orientations respectively, followed by O- and F-surfaces. This could be explained by difference of charge density as shown in FIG. 3B, showing parallel urea adsorption configuration on the surfaces of MXenes. There was a more obvious charge transfer between urea and OH-surface. Meanwhile, the urea adsorption effects on the interlayer spacing of Ti₃C₂Tₓ were calculated. In general, presence of urea in between MXene layers expands the interlayer spacing (FIGS. 3A-3B). As urea might be protonated in acid solution, the interaction between protonated urea and Ti₃C₂Tₓ surfaces was calculated as shown in FIGS. 4A-4B. Protonated urea will decompose on OH-surface, resulting in the formation C—(NH₂)₂ and H₂O, while has enhanced adsorption on O- or F-surface with binding energies of −4.10 and 2.31 eV respectively.

In a word, MXenes were shown to have strong interactions with urea or protonated urea, consistent with MXenes acting as an effective adsorbent of urea.

### Example 6: Expanded Studies—Methods

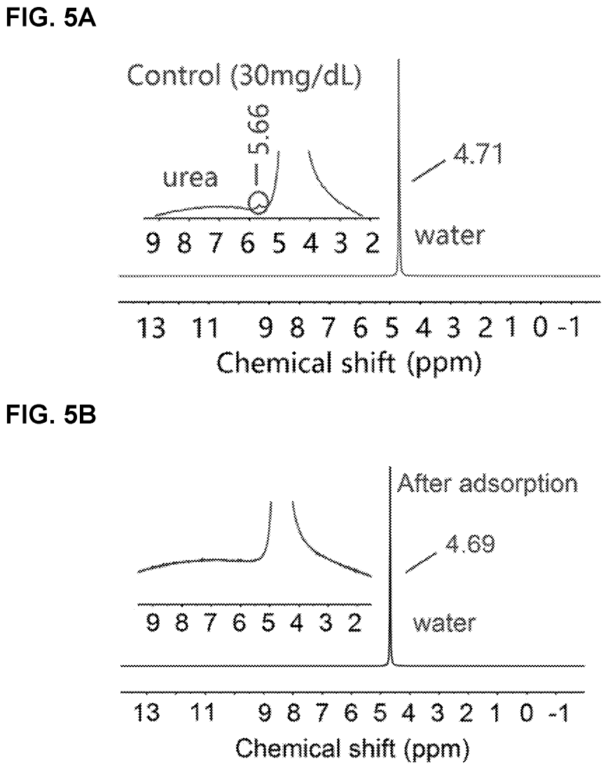

Data from the actual adsorption of urea from aqueous solutions were used to validate these theoretical calculations. To determine the efficiency of urea removal, many techniques have been adopted, such as nuclear magnetic resonance (NMR) technical, high performance liquid chromatography (HPLC) and other chemical reaction coupled colorimetric and fluorometric methods. FIGS. 5A-5D show proton nuclear magnetic resonance (¹H-NMR) spectrum of urea in aqueous solution at low (~30 mg/dL) and high (3,000 mg/dL) concentrations before and after urea adsorption using Ti₃C₂Tₓ. The exchange of protons between urea and water is greatly enhanced by small changes in pH. Therefore, in the presence of MXenes, which are strong Lewis acids, the urea is protonated, resulting in the proton transfer from water to urea which cause the collapse of the two peaks into a single peak. The process of urea protonation is acid-base (Schematic 1); the protonation of urea first happens on the urea acyl oxygen atom and then leads to a second protonation at one of the nitrogen atoms. As a result, the signal of urea disappeared (FIGS. 5B and 5D); however, this is not conclusive evidence of complete urea adsorption by MXenes, because it can be a signal effect caused by the pH change in the solution.

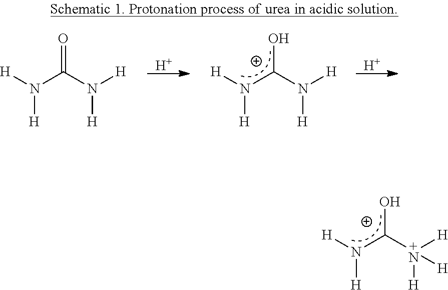

Another possible cause could stem from the terminated OH groups present on the surface of Ti₃C₂Tₓ acting as a nucleophile, attacking the carbonyl carbon atom of the urea molecule (Schematic 2). The acid-base catalyzed reaction proceeds through a tetrahedral intermediate and formed ammonium carbamate, which causes collapse of the two peaks into a singlet. Therefore, the ¹H-NMR measurement was not suitable for quantitative analysis. However, according to previous calculations, —OH moieties have a very strong adsorption on MXene surface with binding energy over −10 eV. Therefore, it is difficult for OH to leave MXene surface. But, it is easy to break O—H bonds.

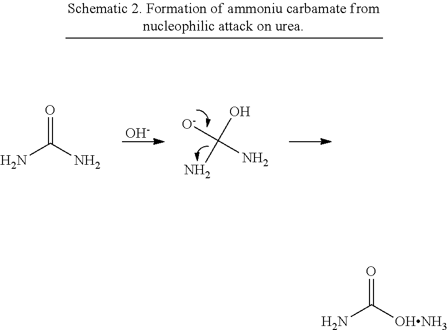

To perform the quantitative analysis of urea removal from aqueous solution, the BioAssay Systems' urea assay kit method was used. FIG. 1A shows the effect of mass-loading of Ti₃C₂Tₓ on adsorption capacity and the comparison of urea removal efficiency from aqueous solution. The urea concentration significantly reduced with increased Ti₃C₂Tₓ mass loading ranging 0.155-5.000 g. The adsorption efficiency by Ti₃C₂Tₓ can reach 98% within 4 min. Even at lower mass-loading of Ti₃C₂Tₓ, 0.155 g, urea removal efficiency was as high as 80% (FIG. 1B). The adsorption capacity based on the kinetic studies is 9.7 mg/g with a lower mass-loading (0.155 g). This was indicative of almost all active sites in the MXenes being occupied. Urea adsorption capacity was likely reduced by the formation of hydrogen bonds between water molecules and the MXene, limiting the active sites available for urea absorption and also by solvation of urea.

The adsorption kinetics of two other MXenes, Ti₂CTₓ and Mo₂TiC₂Tₓ were also investigated and compared with Ti₃C₂Tₓ adsorption (FIG. 1C). The results indicated that all materials with a mass-loading of 0.155 g adsorbed urea rapidly within 4 min, after which, adsorption showed a small change by increasing the contact time indicating that an equilibrium state was obtained. The reason for the fast adsorption process is believed to be due to the presence of larger number of active groups such as —OH, —O and —F on the surface of the MXenes, which can form the hydrogen bonds with urea immediately. The results indicate that Ti₃C₂Tₓ shows the best adsorption performance with the highest urea removal efficiency from aqueous solution of these three MXenes (FIG. 1D).

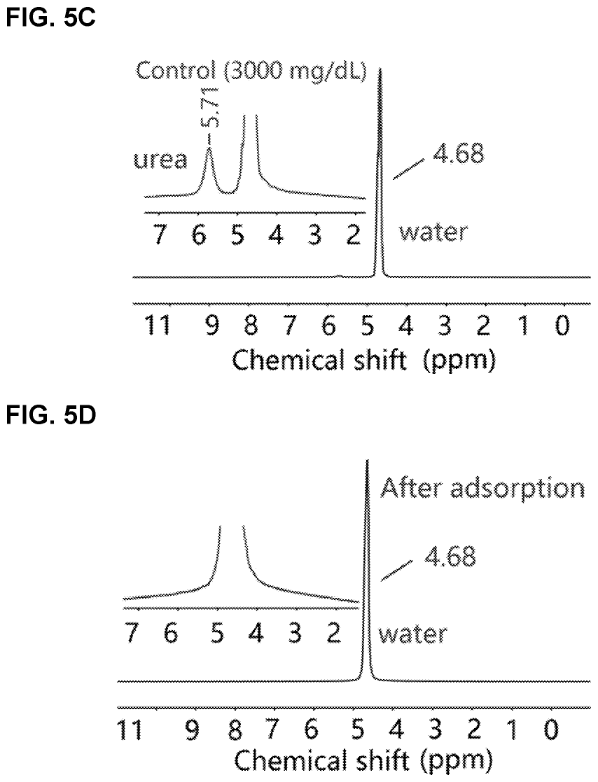

FIG. 6 shows the equilibrium adsorption isotherm of urea on Ti₃C₂Tₓ and fitting using Langmuir, Freundlich and Langmuir-Freundlich models. The adsorption capacity along with the corresponding constants for each model was estimated (Table 1). The Langmuir isotherm theory assumes monolayer coverage of adsorbate over a homogeneous adsorbent surface. Once a site is filled, no further sorption can take place at that site, indicating that the surface reaches a saturation point where the maximum adsorption of the surface will be achieved. The Freundlich expression isotherm theory was used to describe heterogeneous systems, which assumed that as the adsorbate concentration increases the concentration of adsorbate on the adsorbent surface will increase as well. The maximum adsorption capacities (qₒ) of Ti₃C₂Tₓ adsorbent calculated from the Langmuir and Langmuir-Freundlich models were found to be 7.5 mg/g and 10.4 mg/mg, respectively. By comparing the constants in Table 1 for three isotherm models, the Langmuir and Langmuir-Freundlich isotherms show similar adsorption capacity to the experimental data (9.7 mg/g), suggesting that physical sorption plays an important role in this adsorption process. The Langmuir-Freundlich isotherm model presents a better fit with a regression coefficient value (R²=0.9569) higher than Langmuir (R²=0.9233) and Freundlich isotherm (R²=0.9449) models. The maximum adsorption capacity (qₒ) calculated from the Langmuir-Freundlich fit closer to the experimentally determined value than the capacity calculated by other two models, suggesting the heterogeneous adsorption of urea on Ti₃C₂Tₓ in this case. K is a constant which characterizes the strength of adsorbate binding to the adsorbent, while n is the heterogeneity factor indicating, the degree of nonlinearity between solution concentration and adsorption. A value of n<1 indicate adsorption is a physical process.

TABLE 1
Parameters of Langmuir, Freundlich and Langmuir-Freundlich models for
adsorption of urea on Ti₃C₂Tₓ.

| | Langmuir | | | Freundlich | | | Langmuir-Freundlich | | | |
|---|---|---|---|---|---|---|---|---|---|---|
| | (eq. 3) | | | (eq. 4) | | | (eq. 5) | | | |
| | qₒ, | K, | | K, (mg/g) | | | qₒ, | | K, | |
| Adsorbent | mg/g | L/mg | R² | (L/mg)^{1/n} | n | R² | mg/g | n | L/mg | R² |
| Ti₃C₂Tₓ | 7.5 | 0.069 | 0.9233 | 2.35 | 0.21 | 0.9449 | 10.4 | 0.46 | 0.026 | 0.9569 |

### Example 7: Expanded Studies—Adsorption of Urea from Aqueous Solution

The urea adsorption of various MXenes was tested using aqueous solutions at ambient conditions. The initial concentration of urea in aqueous solution was ~30 mg/dL, which corresponded to the normal urea concentration in the dialysate of a patient suffering from uremia. To study the kinetics and removal efficiency, different mass-loadings of MXene (Ti₃C₂Tₓ) powder (5.000, 2.500, 1.250, 0.625, 0.312 and 0.155 g−adsorbent dosage) were added into 6 mL of urea aqueous solution mixed by hand shaking (3 min) and then held static. For Mo₂TiC₂Tₓ and Ti₂CTₓ materials the adsorbent dosage was 0.625 g. At 4, 9, 18, 33, and 63 min time points, the urea solutions were sampled (1 mL) using a micropipette and then centrifuged at 14,000 rpm. Afterwards, the supernatants were collected and centrifuged again (14,000 rpm) to remove small particles of adsorbent prior to analysis.

The adsorption isotherm of urea from aqueous solution was conducted only for Ti₃C₂Tₓ which preliminary kinetic studies showed the highest removal efficiency among all MXenes studied here. The same amount of Ti₃C₂Tₓ (0.625 g) was weighted and added to urea solutions (6 mL) ranging in concentration from 30 to 450 mg/dL. After reaching equilibrium (60 min), the samples were centrifuged, supernatants were analyzed for their urea content, and the equilibrium adsorption isotherm was constructed. Linear detection ranged from 0.08 mg/dL (13 μM) to 100 mg/dL (17 mM) urea in a 96-well plate assay. Therefore, the initial and final concentration of urea solution was tested with a dilution.

The concentration of urea (in mg/dL) was determined using BioAssay Systems' urea assay kit (DIUR-100) by reading the optical density (OD) at 520 nm following Equation 1:

$$[ Urea ] = \frac{{OD}_{sample} - {OB}_{blank}}{{OD}_{standard} - {OD}_{blank}} \times n \times [ STD ] \qquad ( 1 )$$

where ODₛₐₘₚₗₑ, OD_{blank} and OD_{standard} are OD values of sample, standard and water, respectively. The variable n is the dilution factor and [STD]=50 (or 5 for low urea samples) was the urea standard concentration (in mg/dL).

The amount adsorbed urea was calculated from Equation 2:

$$q = \frac{( {C}_{o} - {C}_{e} ) \times V}{m} \qquad ( 2 )$$

where q was the amount of adsorbed urea (mg/g), Cₒ is the initial concentration of solute (mg/dL), Cₑ is the final concentration of solute at equilibrium (mg/dL), V is the volume aliquot adsorbate (mL) and m is the mass of adsorbent (g).

The Langmuir, Freundlich, and Langmuir-Freundlich adsorption isotherm equations were employed to fit experimental adsorption data. The equations of Langmuir, Freundlich and Langmuir-Freundlich isotherms are shown in Equations 3, 4, and 5 respectively

$$\frac{{q}_{e}}{{q}_{o}} = \frac{{KC}_{e}}{( 1 + {KC}_{e} )} \qquad ( 3 )$$

$$q = {KC}^{n} \qquad ( 4 )$$

$$\frac{{q}_{e}}{{q}_{o}} = \frac{{( {KC}_{e} )}^{n}}{1 + {( {KC}_{e} )}^{n}} \qquad ( 5 )$$

where qₑ is the adsorbed amount of urea per gram of adsorbent at equilibrium, qₒ is the maximum adsorption of urea per gram of the adsorbent, K is the Langmuir-type constant defined by the Van′t Hoff equation, and the exponential term n represents the heterogeneity of the site energies.

### Example 8: Adsorption of Urea from Dialysate

The urea removal adsorption efficiency was tested in dialysate of uremic patients directly. The method of testing was the same as described above for aqueous solutions. The concentration of urea (in mg/dL) was determined using BioAssay Systems' urea assay kit.

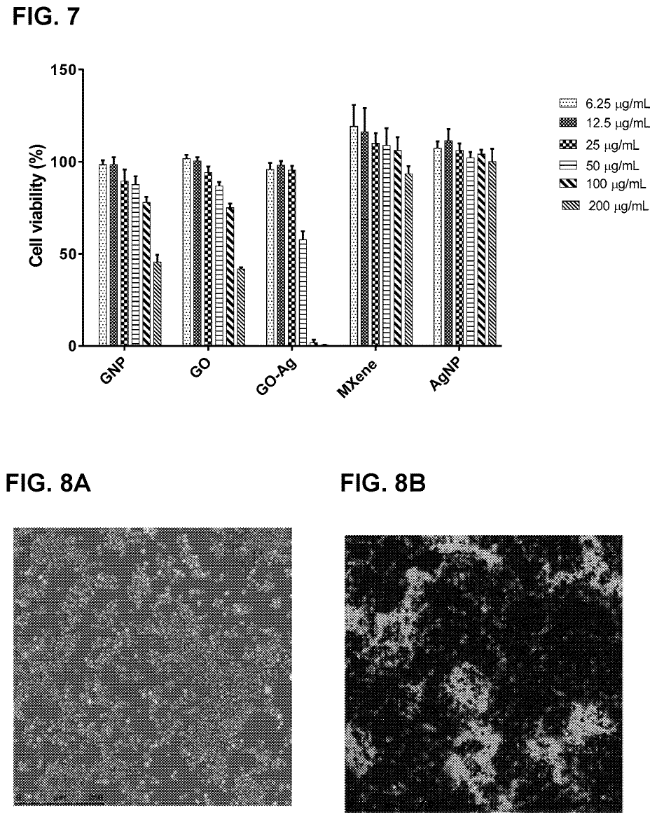

FIG. 2A shows the effect of mass-loading of Ti₃C₂Tₓ on the removal efficiency of urea in dialysate. The urea concentration in dialysate reduces significantly for the mass loading of both 2.500 g and 5.000 g. For these two mass loadings, the removal efficiency of urea for Ti₃C₂Tₓ is 94%, while at lower mass loading, it reduced to 15% (FIG. 8B). For 5.000 and 2.500 g Ti₃C₂Tₓ mass loadings, urea adsorption efficiency did not change significantly from dialysate (94%) to aqueous solution (98%). However, at lower mass loadings, the removal efficiency of urea from dialysate decreased significantly compared to the adsorption of urea from aqueous solution. Moreover, the adsorption capacity of urea from dialysate reduced to 1.6 mg/g, a significant decrease compared to the adsorption of urea from aqueous solution (9.7 mg/g) for the same mass-loading (0.155 g). It is worth mentioning that the adsorption capacity of urea from dialysate increased rapidly in the first 4 min, after which it slightly increases (FIG. 2C). The lower adsorption capacity in dialysate compared to aqueous solution (compare FIG. 1C and FIG. 2C) could be related to replacement of adsorbed competing molecules in the dialysate, i.e., the ions in the dialysate compete with urea to occupy the adsorption sites. Similar adsorption kinetic behavior was observed for all three MXenes (FIG. 2C) with a fast and efficient removal within 4 min. Of those tested, the best MXene adsorbent for urea from dialysate was Ti₃C₂Tₓ, which outperformed Ti₂CTₓ and Mo₂TiC₂Tₓ (FIG. 2D). In general, adsorption capacities of these MXenes were comparatively low. However, in adsorption processes the kinetics of adsorption was as important as adsorption capacity, therefore, the performance of a WAK could be improved by decreasing the time of the adsorption.

### Example 9: Cytotoxicity Assessment of MXene Ti₃C₂Tₓ

A stock suspension (1 mg/mL) of each nanomaterial (GNP, GO, GO-Ag, MXene and AgNP) in media was sonicated for 30 minutes and diluted 1:2.5 in media to a concentration of 400 μg/mL. 3T3 cells were seeded in a 96-well plate at a density of 1×10⁴ cells/well and incubated for 24 hours at 37 °C. Spent medium was removed and 100 μL of fresh medium was added to each well. 100 μL of each nanomaterial suspension was added to triplicate wells at varying concentrations (6.25, 12.5, 25, 50, 100 and 200 μg/mL) and plates were incubated for 24 hours at 37 °C, 5% CO₂. The NP suspension was removed, and wells were washed with sterile phosphate buffered saline. MTS reagent was diluted 1 in 6 in media, and 120 μL was added to each well. The plates were wrapped in foil and incubated for 2 hours at 37 °C, 5% CO₂. The reagent was then transferred to a fresh 96 well plate to avoid interference of any NPs adhered to the cell surface on the base of the wells since the NPs tended to increase the absorbance readings. Absorbance was read on a BioRad plate reader at a wavelength of 490 nm. The live-dead stain was prepared in PBS to a concentration of 1 μM, and 100 μL of solution was added to each of the wells of the 96 plates prepared as for the MTS assay. Fluorescent imaging was carried out using confocal microscopy. Statistical analysis was conducted on Graphpad Prism software and a two-way ANOVA of % cell viability.

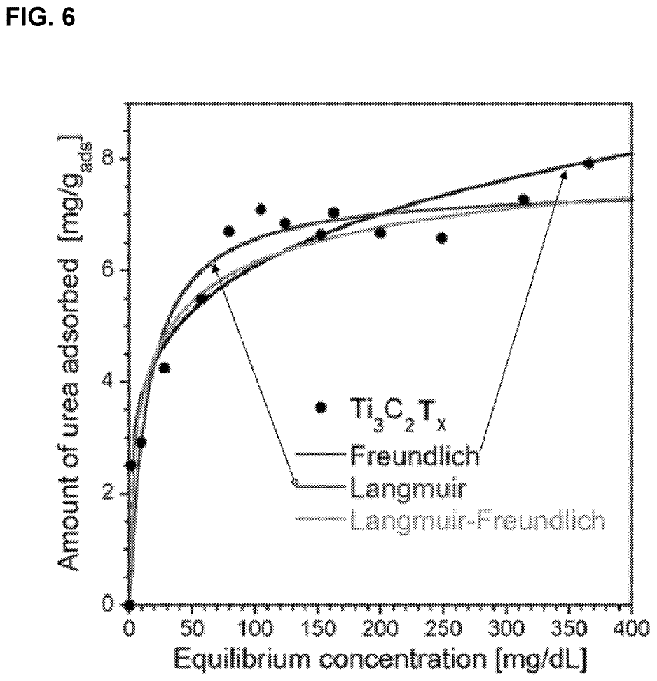

In the concentration range tested, MXene had no impact on 3T3 cell metabolism as indicated in FIG. 7 and FIG. 8A-B. In contrast GO-Ag induced a significant reduction in cell viability at concentrations of 50 μg/mL and above. GO and GNP also produced a significant reduction in cell viability at concentrations of 200 μg/ml. AgNP did not have a significant impact on cell metabolism in the concentration range used.

In this way, MXenes have been shown to be the first synthetic material that can completely remove urea from blood/dialysate at the clinical levels. Due to its very strong sorption capability, very small amounts of MXene are needed to remove urea by adsorptive process.

As those skilled in the art will appreciate, numerous modifications and variations of the present invention are possible in light of these teachings, and all such are contemplated hereby.

All references cited within this specification are incorporated by reference in their entireties for all purposes, or at least for their teachings in the context of their recitation.

1. A dialysis system, comprising:
an inlet adapted to receive an initial aqueous urea solution;
a cartridge for removing urea from the initial aqueous urea solution,
the cartridge comprising a MXene composition to which the system directs the initial aqueous urea solution, the cartridge adapted to allow the initial aqueous urea solution to contact the MXene composition contained in the cartridge,
the dialysis system configured for placing the inlet into fluid communication with the cartridge.

2. The dialysis system of claim 1, wherein the MXene composition is comprised in a permeable bed.

3. The dialysis system of claim 1, wherein the system is portable.

4. The dialysis system of claim 1, wherein the system is operable in a through-pass mode.

5. The dialysis system of claim 1, wherein the system is operable in a recycle mode.

6. The dialysis system of claim 1, wherein the MXene composition comprises a composition comprising at least one layer having first and second surfaces, each layer described by a formula Mₙ₊₁XₙTₓ, such that
each X is position within an octahedral array of M, wherein
M is at least on Group IIIB, IVB, VB, or VIB metal, wherein
each X is C, N, or a combination thereof,
n=1, 2, or 3; and wherein
Tₓ represents surface termination groups.

7. The dialysis system of claim 6, wherein a surface termination group comprises alkoxide, carboxylate, halide, hydroxide, hydride, oxide, sub-oxide, nitride, sub-nitride, sulfide, thiol, or a combination thereof.

8. The dialysis system of claim 6, wherein M is at least one group IVB, Group VB, or Group VIB metal.

9. The dialysis system of claim 6, wherein M is Ti, Mo, Nb, V or Ta, or a combination thereof.

10. The dialysis system of claim 6, wherein Mₙ₊₁Xₙ is Ti₂C, Mo₂Ti₂C, Mo₂TiC₂, Ti₃C₂, or a combination thereof.

11. The dialysis system of claim 1, wherein the MXene composition comprises a composition comprising at least one layer having first and second surfaces, each layer comprising:
a. a substantially two-dimensional array of crystal cells,
b. each crystal cell having an empirical formula of M′₂M″ₙXₙ₊₁, such that each X is positioned within an octahedral array of M′ and M″, and where M″₂ are present as individual two-dimensional array of atoms intercalated between a pair of two-dimensional arrays of M′ atoms,
c. wherein M′ and M″ are different Group IIIB, IVB, VB, or VIB metals,
d. wherein each X is C, N, or a combination thereof; and
e. n=1 or 2.

12. The dialysis system of claim 11, wherein n is 1, M′ is Mo, and M″ is Nb, Ta, Ti, or V, or a combination thereof.

13. The dialysis system of claim 11, wherein n is 2, M′ is Mo, Ti, V, or a combination thereof, and M″ is Cr, Nb, Ta, Ti, or V, or a combination thereof.

14. The dialysis system of claim 11, wherein M′₂M″ₙX+1 comprises Mo₂TiC₂, Mo₂VC₂, Mo₂TaC₂, Mo₂NbC₂, Mo₂Ti₂C₃, Cr₂TiC₂, Cr₂VC₂, Cr₂TaC₂, Cr₂NbC₂, Ti₂NbC₂, Ti₂TaC₂, V₂TaC₂, Mo₂Ti₂C₃, Mo₂V₂C₃, Mo₂Nb₂C₃, Mo₂Ta₁C₃, Cr₂TiC₂, Cr₂VC₂, Cr₂Nb₂C₃, Cr₂Ta₂C₃, Nb₂Ta₂C₃, Ti₂Nb₂C₃, Ti₂Ta₂C₃, V₂Ta₂C₃, V₂Nb₂C₃, or V₂Ti₂C₃, or a nitride or carbonitride analog thereof.

15. The dialysis system of claim 11, wherein the MXene composition comprises a plurality of stacked layers.

16. The dialysis system of claim 11, wherein at least one of said surfaces of each layer has surface terminations comprising alkoxide, carboxylate, halide, hydroxide, hydride, oxide, sub-oxide, nitride, sub-nitride, sulfide, thiol, or a combination thereof.

17. A method, comprising:
causing introduction of an initial aqueous urea solution to a dialysis system according to claim 1 such that the initial aqueous urea solution contacts the MXene composition of the cartridge,
contact between the initial aqueous urea solution and the MXene composition being at ambient or near ambient temperatures and under such conditions that urea in the initial aqueous urea solution is reduced from an initial concentration in the initial aqueous urea solution to a final concentration in a final solution, wherein the initial concentration of urea in the initial aqueous urea solution is in a range of from 10 mmol/L to 1000 mmol/L, or is initially in a concentration range from 15 to 40 mg/dL mg/dL, and the final concentration is at least 10% less than the initial concentration, and
wherein the initial aqueous urea solution is or comprises blood or a blood product and the ambient or near ambient temperatures and conditions used do not compromise the utility of the blood or blood product for later use by a human patient.

18. The method of claim 17, wherein the final concentration is at least 50% less than the initial concentration.

19. The method of claim 17, wherein the contacting is for less than about 5 minutes.

20. The method of claim 17, wherein adsorption of the urea to the MXene attains equilibrium within 5 minutes.
